(19) United States
(12) Patent Application Publication
(10) Pub. No.: US 2005/0095577 A1
(43) Pub. Date: May 5, 2005

(54) Protein bioarray on silane-modified substrate surface

(76) Inventors: Dan-Hui Dorothy Yang, Sunnyvale, CA (US); Magdalena Anna Bynum, San Jose, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/698,225

(22) Filed: Oct. 31, 2003

(51) Int. Cl. C12Q 1/00

(52) U.S. Cl. 435/4

(57) ABSTRACT

The invention provides a method of producing a protein bioarray that includes providing a substrate comprising a solid support and a surface modification layer bound to the solid support. The surface modification layer includes a first moiety having the structure —Si—R¹ and a second moiety having the structure —Si-L-R², wherein R¹ is a chemically inert moiety selected from the group consisting of C₃ to C₃₀ alkyl and benzyl optionally substituted with 1 to 5 halogen atoms, L is a linking group, and R² is a chemically inert hydrophilic moiety. The method of producing the protein bioarray further includes providing at least two solutions, wherein each solution contains a probe protein. In the method, each of the solutions is then deposited at its own discrete site on the substrate. The probe proteins deposited on the substrate become non-covalently bound to the substrate.

### FIELD OF THE INVENTION

The invention relates generally to bioarrays, which are useful in analyte detection assays, and other applications. More specifically, the invention relates to protein bioarrays on silane-modified substrates.

### BACKGROUND OF THE INVENTION

As genomic research progresses from the study of genes to the proteins they encode, the challenge of deciphering protein expression and function on a genome-wide scale becomes more difficult. High throughput detection of proteins and their interaction with DNAs and biological active molecules are becoming increasingly important in drug discovery, medicine, and biological research. In recent years, standard molecular biology techniques have begun the transition to the development of array based sensing, either in micro-well plate format or on solid substrate. Haab et al. (2001) Genome Biology, 2(2), 1-13; MacBeath et al. (2000) Science, 289: 1760-63. Microarray analysis of proteins provides features such as high throughput, low sample consumption, and the potential for highly accurate and sensitive detection in multiple wavelength regions using scan fluorescence microscopy. Protein microarray analysis presents numerous applications in biology and medicine. Protein profiling, protein-protein interactions, protein-small molecule interactions, and kinase activity and function are among the examples of current developments. See MacBeath (2001) Nature Biotechnology, 19: 828-29; and references therein. Noticeably, antibody microarrays are used for prostate cancer marker discovery by comparing serum protein profiles of cancer patients and healthy people in a two-color assay system. Miller et al. (2001) Disease Markers 17: 225-34. Such research is expected to lead to new diagnostic assays for disease and also provide new research tools for studying changes in protein expression pattern during the course of a disease. Such assays and tools provide researchers with additional resources in elucidating disease mechanisms and in developing therapies targeted to the specific molecular cause of the disorder.

The acquisition of high specificity and high affinity protein probes poses a challenge to obtaining useful protein microarray data. A broad range of probes are available, including mono- and polyclonal antibodies, antibody fragments, alternative protein-binding scaffold and DNA-based aptamers. Another technical challenge is the development of surface chemistry for probe deposition. In the microarray format, the protein probes are spotted onto chemically modified solid surfaces at high spatial densities. Since proteins have an almost unlimited variety of charges, polarity and structures, efficient attachment of specific spotted proteins while repelling the adsorption of non-specific background proteins can be difficult. The development of surface chemistry that allows strong binding of the probes to the surface and also allows accessibility of a target ligand to the surface-bound probes is essential. The microarray surface material and method of protein deposition typically has a profound influence on the overall efficiency of protein immobilization and protein activity preservation.

Two different mechanisms are typically employed for deposition of probes on a microarray surface: (1) physical adsorption; and (2) covalent attachment, generally via the reaction of amine or thiol with the functionalized surface. Polylysine (PLL) is a common surface for retaining probes through non-specific molecular interactions. Haab et al. (2001) Genome Biology, 2(2), 1-13. PLL is positively charged at physiological pH. Probes are retained on the PLL surface by charge-charge interactions, hydrophobic-hydrophobic interactions as well as hydrogen bonding. However, PLL places some constraints on the assay conditions because certain conditions would be more likely to wash away probes. There are some commercial surfaces such as Motorola 3D-link™ to covalently bind probes in a basic pH environment. This surface is optimized for DNA microarrays with possible application for protein deposition. Chemical reaction of DNAs or proteins requires humid or liquid conditions and the reaction is slow, especially for higher molecular weight proteins. Another commercial surface is the salicylhydroxamic (SHA) modified glass substrate. It chelates with phenyldiboronic acid modified probes at neutral pH, allowing effective immobilization of proteins and other macromolecules (Prolinx, Inc. Protocol #VMT2000). Unfortunately, this requires the modification of every probe and presents difficulties in a high throughput format. Another method appears to be the attachment of biotinylated proteins through a streptavidin-biotin bridge on the end of poly(ethylene glycol) (PEG) polymer strand. Ruiz-Taylor et al. (2001) Proc. Natl. Acad. Sci. 98, 852-57. The PEG, which is attached to PLL coating on the glass, efficiently repels non-specific background protein, yet specific attachment of protein probes is achieved by the biotin-streptavidin junction. However, this surface also requires the modification of spotted probes with biotin. Zyomyx (U.S. Pat. No. 6,365,418; U.S. Pat. No. 6,406,921) has patents on the mixed surface of hydrophobic self-assembled monolayer linked to hydrophilic self-assembled monolayer on the top. Such a patterned surface is aimed to reduce non-specific protein interaction during target binding. The preparation of such surfaces presents considerable technique challenges and higher cost. Also, the patterned area has to be precise so that writing apparatus can be perfectly aligned with the pattern. This could be a major issue in manufacturing smaller features for the purpose of making high-density array. Thus, there is a demand to develop a low-cost surface for efficient and high-throughput probe deposition simple enough for common laboratory practice.

Another important application for chemically modified surface is for solid phase synthesis. Initial derivatization of a substrate surface enables the synthesis of polymers such as peptides and oliginucleotides. For example, the modification of a glass substrate is typically based on the reaction with chlorosilane or alkyloxysilane. Lefkowitz et al. (U.S. Pat. No. 6,258,454 B1; U.S. Pat. No. 6,444,268 B2 have described a general method of preparing hydrophobic self-assembled surface with different functionality by adjusting the ratio of two silanes. The hydroxyl group on the surface provides the anchor for in-situ oligonucleotide synthesis by reacting with phosphoramidite-modified nucleotide.

What is needed is a convenient and effective method of making protein bioarrays.

### SUMMARY OF THE INVENTION

The invention addresses the aforementioned deficiencies in the art, and provides novel methods for making protein bioarrays. In short, the method involves providing a substrate having a surface modification layer, providing at least two solutions containing the probe proteins, and depositing each solution at a different discrete site on the substrate to produce the protein array.

In a typical embodiment in accordance with the invention, a method of producing a protein bioarray includes providing a substrate comprising a solid support and a surface modification layer bound to the solid support The surface modification layer includes a first moiety having the structure —Si—R¹ and a second moiety having the structure —Si-L-R², wherein R¹ is a chemically inert moiety selected from the group consisting of C₃ to C₃₀ alkyl and benzyl optionally substituted with 1 to 5 halogen atoms, L is a linking group, and R² is a chemically inert hydrophilic moiety. The method of producing the protein bioarray further includes providing at least two solutions, wherein each solution contains a probe protein. In the method, each of the solutions is then deposited at its own discrete site on the substrate. The probe proteins deposited on the substrate become non-covalently bound to the substrate.

In a typical embodiment, the solutions are allowed to dry on the substrate. In certain embodiments, at least one solution contains a different probe protein than another of the solutions so that the features formed therefrom will display different probes. In certain embodiments, at least two solutions may contain the same probe protein so that the features formed at the respective sites of the two solutions will display the same probe. In some embodiments, at least two solutions contain the same probe protein, but at different concentrations to result in the probe bound to the substrate at different concentrations at at least two discrete sites.

In some embodiments, the method further includes contacting the substrate surface with a blocking composition after the probe proteins are non-covalently bound to the substrate. In some embodiments the discrete sites on the substrate are separated by intervening space, and the blocking composition is generally washed over a surface of the substrate, thereby contacting the discrete spots and the intervening sites on the substrate. The blocking composition typically contains one or more blocking proteins, such that the blocking proteins typically non-covalently bind to portions of the substrate surface not already occupied by probe proteins. The binding of blocking proteins on the substrate surface tends to block sites/areas that non-specifically bind to proteins and would therefore contribute to noise in assays employing the protein array.

In an additional embodiment, the invention provides a protein array that includes a substrate comprising a solid support and a surface modification layer bound to the solid support. The surface modification layer includes a first moiety having the structure —Si—R¹ and a second moiety having the structure —Si-L-R², wherein R¹ is a chemically inert moiety selected from the group consisting of C₃ to C₃₀ alkyl and benzyl optionally substituted with 1 to 5 halogen atoms, L is a linking group, R² is a chemically inert hydrophilic moiety. The protein array further includes a plurality of discrete sites on the substrate each site having a probe protein bound, thereto via non-covalent interaction. In particular embodiments, the protein array further includes blocking protein bound to the substrate.

Additional objects, advantages, and novel features of this invention shall be set forth in part in the descriptions and examples that follow and in part will become apparent to those skilled in the art upon examination of the following specifications or may be learned by the practice of the invention. The objects and advantages of the invention may be realized and attained by means of the combinations, compositions and methods particularly pointed out in the appended claims.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein
FIG. 1 schematically illustrates a substrate comprising a solid support and a surface modification layer, the substrate having multiple protein arrays formed thereon.

FIG. 2 depicts a portion of a single array having features at discrete sites on a substrate which comprises a solid support and a surface modification layer.

To facilitate understanding, identical reference numerals have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “an insoluble support” includes a plurality of insoluble supports. Also, reference to “first moiety having the structure —Si—R¹” includes mixtures of moieties having the recited structure, while, similarly, “a second moiety having the structure —Si-L-R²” includes mixtures of such moieties. In this specification and in the claims that follow, reference will be made to a number of terms that shall be defined to have the following meanings unless a contrary intention is apparent.

An “array” includes any one, two or three dimensional arrangement of addressable regions bearing a particular chemical moiety or moieties (for example, polynucleotide sequences) associated with that region. A “bioarray” is an array of biomolecules. “Biomolecule” refers to molecules generally derivable from living organisms, or analogues thereof. Biomolecules include, e.g. amino acids, oligopeptides, polypeptides, nucleotide monomers, oligonucleotides, polynucleotides, saccharides, polysaccharides, hormones, growth factors, peptidoglycans, or the like, or analogues thereof. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). “Probe protein” may be used herein to refer to a protein that is intended to be bound to a substrate to serve as a probe. An “array format” refers to one or more characteristics of the array, such as feature positioning, feature size, and some indication of a moiety at a given location. “Feature deposition” refers to a process of putting biomolecules on the substrate surface after the surface is prepared: feature deposition encompasses, e.g. placing droplets of biomolecules on the surface. Various methods are readily available in the art and may be routinely adapted to use with the method and apparatus of the current invention by one of ordinary skill in the art. “Binding assay” references a process of contacting a bioarray with a mobile phase containing target moieties. A “blocking composition” is a composition that bonds preferentially to surface moieties and reduces background signal and/or reduces the number of sites available for non-specific binding to occur. Non-specific binding results from binding of sample (or “target”) molecules at sites other than the intended feature or unintended binding of sample molecules at a feature. “Passivation” refers to any process of chemically modifying the surface of a substrate, e.g. to block non-specific binding.

As used herein, “chemically inert” means that the group referred to as “chemically inert” will not react to form a covalent bond with a protein under conditions used (1) in contacting a surface bearing the chemically inert group with a solution containing a probe protein to form a protein bioarray according to the method described herein, (2) in contacting a surface bearing the chemically inert group with a blocking composition comprising a blocking protein, and/or (3) in performing a binding assay using a protein array provided in the current invention.

“Chemically inert” hydrophilic groups means groups that are hydrophilic in character and meet the above definition of “chemically inert”.

The “surface energy” γ (measured in ergs/cm.sup.2) of a liquid or solid substance pertains to the free energy of a molecule on the surface of the substance, which is necessarily higher than the free energy of a molecule contained in the in the interior of the substance; surface molecules have an energy roughly 25% above that of interior molecules. The term “surface tension” refers to the tensile force tending to draw surface molecules together, and although measured in different units (as the rate of increase of surface energy with area, in dynes/cm), is numerically equivalent to the corresponding surface energy. By modifying a substrate surface to “reduce” surface energy is meant lowering the surface energy below that of the unmodified surface.

The term “ligand” as used herein refers to a moiety that is capable of covalently or otherwise chemically binding a compound of interest. However, the term “ligand” as used herein may also refer to a compound that is not synthesized on the novel functionalized substrate, but that is “pre-synthesized” or obtained commercially, and then attached to the substrate.

The term “sample” as used herein relates to a material or mixture of materials, typically, although not necessarily, in fluid form, containing one or more components of interest.

As used herein, “protein” references a compound having a series of amino acid subunits bound via peptide bonds; the protein may have from 2 to 1000 or more amino acid subunits. “Peptide” references a compound having a series of amino acid subunits bound via peptide bonds, wherein the compound has from about 2 to about 50 amino acid subunits, more typically from about 2 to about 30 amino acid subunits, still more typically from about 3 to about 20 amino acid subunits. “Amino acid” references an amphoteric compound containing an amino group and a carboxylic acid group; typical examples include the alpha amino acids that typically make up proteins.

“Moiety” and “group” are used to refer to a portion of a molecule, typically having a particular functional or structural feature, e.g. a linking group (a portion of a molecule connecting two other portions of the molecule), or an ethyl moiety (a portion of a molecule with a structure closely related to ethane). “Residue” is sometimes used herein to reference a moiety that is a subunit of a larger moiety having a plurality of the subunits joined together.

“Linkage” as used herein refers to a first moiety bonded to two other moieties, wherein the two other moieties are linked via the first moiety. Typical linkages include ether (—O—), oxo (—C(O)—), amino (—NH—), amido (—N—C(O)—), thio (—S—), phospho (—P—), ester (—O—C(O)—).

“Bound” may be used herein to indicate direct or indirect attachment. In the context of chemical structures, “bound” (or “bonded”) may refer to the existence of a chemical bond directly joining two moieties or indirectly joining two moieties (e.g. via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and also embodiments where the attachment is indirect. As used herein with reference to the protein non-covalently bound to the solid support, “non-covalently bound” means that the protein is bonded to the substrate (e.g. the modification layer) by other than covalent means, including ionic, van der Waals, and hydrogen bonding.

By “protecting group” as used herein is meant a moiety which prevents a portion of a molecule from undergoing a chemical reaction under specified conditions, but which is removable from the molecule following exposure of the molecule to the specified conditions; the protecting group thus allows an unprotected portion of a molecule to undergo a chemical reaction under the specified conditions while preventing the protected portion of the molecule from undergoing a chemical reaction. This is in contrast to a “capping group,” which permanently binds to a segment of a molecule to prevent any further chemical transformation of that segment.

The term “functionalization” as used herein relates to modification of a solid substrate to provide a plurality of functional groups on the substrate surface. By a “functionalized surface” as used herein is meant a substrate surface that has been modified so that a plurality of functional groups are present thereon.

“Functionalized” references a process whereby a material is modified to have a. specific moiety bound to the material, e.g. a molecule or substrate is modified to have the specific moiety; the material (e.g. molecule or support) that has been so modified is referred to as a functionalized material (e.g. functionalized molecule or functionalized support).

The term “substituted” as used to describe chemical structures, groups, or moieties, refers to the structure, group, or moiety comprising one or more substituents. As used herein, in cases in which a first group is “substituted with” a second group, the second group is attached to the first group whereby a moiety of the first group (typically a hydrogen) is replaced by the second group.

“Substituent” references a group that replaces another group in a chemical structure. Typical substituents include nonhydrogen atoms (e.g. halogens), functional groups (such as, but not limited to amino, sulfhydryl, carbonyl, hydroxyl, alkoxy, carboxyl, silyl, silyloxy, phosphate and the like), hydrocarbyl groups, and hydrocarbyl groups substituted with one or more heteroatoms. Exemplary substituents include alkyl, lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, boronyl, modified alkyl, and modified lower alkyl.

A “group” may include substituted and unsubstituted forms, where context permits. Typical substituents include one or more lower alkyl, modified alkyl, any halogen, hydroxy, or aryl. Any substituents are typically chosen so as not to substantially adversely affect reaction yield (for example, not lower it by more than 20% (or 10%, or 5% or 1%) of the yield otherwise obtained without a particular substituent or substituent combination).

The term “halo” or “halogen” is used in its conventional sense to refer to a chloro, bromo, fluoro or iodo substituent.

The term “alkyl” as used herein, unless otherwise specified, refers to a saturated straight chain, branched or cyclic hydrocarbon group of 1 to 30, typically 1 to 12, carbon atoms, such as methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “lower alkyl” intends an alkyl group of one to six carbon atoms, and includes, for example, methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “cycloalkyl” refers to cyclic alkyl groups such as cyclopropyl, cyclobutyl, cyclopentyl, cyclohexyl, cycloheptyl and cyclooctyl. An alkyl group may be substituted or unsubstituted.

The term “modified alkyl” refers to an alkyl group having from 1 to 30 carbon atoms, and further having additional groups, such as one or more linkages selected from ether-, thio-, amino-, phospho-, oxo-, ester-, and amido-, and/or being substituted with one or more additional groups including lower alkyl, aryl, alkoxy, thioalkyl, hydroxyl, amino, sulfonyl, thio, mercapto, imino, halo, cyano, nitro, nitroso, azido, carboxy, sulfide, sulfone, sulfoxy, phosphoryl, silyl, silyloxy, and boronyl. The term “modified lower alkyl” refers to an alkyl group having from one to six carbon atoms and further having additional groups, such as one or more linkages selected from ether-, thio-, amino-, phospho-, keto-, ester-, and amido-, and/or being substituted with one or more groups including lower alkyl; aryl, alkoxy, thioalkyl, hydroxyl, amino, sulfonyl, thio, mercapto, imino, halo, cyano, nitro, nitroso, azido, carboxy, sulfide, sulfone, sulfoxy, phosphoryl, silyl, silyloxy, and boronyl. In particular embodiments, a modified alkyl group may include from one to about three substituents.

The term “alkoxy” as used herein refers to a substituent —O—R wherein R is alkyl as defined above. The term “lower alkoxy” refers to such a group wherein R is lower alkyl. The term “thioalkyl” as used herein refers to a substituent —S—R wherein R is alkyl as defined above. A haloalkyl group refers to an alkyl group that is substituted with one or more halogen atoms.

The term “alkenyl” as used herein, unless otherwise specified, refers to a branched, unbranched or cyclic (e.g. in the case of C5 and C6) hydrocarbon group of 2 to 30, typically 2 to 12, carbon atoms containing at least one double bond, such as ethenyl, vinyl, allyl, octenyl, decenyl, and the like. The term “lower alkenyl” intends an alkenyl group of two to six carbon atoms, and specifically includes vinyl and allyl. The term “cycloalkenyl” refers to cyclic alkenyl groups.

The term “alkynyl” as used herein, unless otherwise specified, refers to a branched or unbranched hydrocarbon group of 2 to 30, typically 2 to 12, carbon atoms containing at least one triple bond, such as acetylenyl, ethynyl, n-propynyl, isopropynyl, n-butynyl, isobutynyl, t-butynyl, octynyl, decynyl and the like. The term “lower alkynyl” intends an alkynyl group of two to six carbon atoms, and includes, for example, acetylenyl and propynyl, and the term “cycloalkynyl” refers to cyclic alkynyl groups.

The term “aryl” as used herein refers to an aromatic species containing 1 to 5 aromatic rings, either fused or linked, and either unsubstituted or substituted with 1 or more substituents typically selected from the group consisting of lower alkyl, modified lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl. Typical aryl groups contain 1 to 3 fused aromatic rings, and more typical aryl groups contain 1 aromatic ring or 2 fused aromatic rings. Aromatic groups herein may or may not be heterocyclic. The term “aralkyl” intends a moiety containing both alkyl and aryl species, typically containing less than about 24 carbon atoms, and more typically less than about 12 carbon atoms in the alkyl segment of the moiety, and typically containing 1 to 5 aromatic rings. The term “aralkyl” will usually be used to refer to aryl-substituted alkyl groups. The term “aralkenyl” will be used in a similar manner to refer to moieties containing both alkenyl and aryl species, typically containing less than about 24 carbon atoms in the alkenyl portion and 1 to 5 aromatic rings in the aryl portion, and typically aryl-substituted alkenyl. Exemplary aralkyl groups have the structure —(CH2)j-Ar wherein j is an integer in the range of 1 to 24, more typically 1 to 6, and Ar is a monocyclic aryl moiety.

The term “heterocyclic” refers to a five- or six-membered monocyclic structure or to an eight- to eleven-membered bicyclic structure which is either saturated or unsaturated. The heterocyclic groups herein may be aliphatic or aromatic. Each heterocyclic group consists of carbon atoms and from one to four heteroatoms selected from the group consisting of nitrogen, oxygen and sulfur. As used herein, the term “nitrogen heteroatoms” includes any oxidized form of nitrogen, and the quatemized form of nitrogen. The term “sulfur heteroatoms” includes any oxidized form of sulfur. Examples of heterocyclic groups include purine, pyrimidine, piperidinyl, morpholinyl and pyrrolidinyl. Heterocylic groups may be substituted or unsubstituted.

The term “heteroaryl,” as used herein, means an aromatic heterocycle which contains 1, 2, 3 or 4 heteroatoms selected from nitrogen, sulfur or oxygen. A heteroaryl may be fused to one or two rings, such as a cycloalkyl, a heterocycloalkyl, an aryl, or a heteroaryl. The point of attachment of a heteroaryl to a molecule may be on the heteroaryl, cycloalkyl, heterocycloalkyl or aryl ring, and the heteroaryl group may be attached through carbon or a heteroatom. Suitable heteroaryl groups include imidazolyl, furyl, pyrrolyl, thienyl, oxazolyl, thiazolyl, isoxazolyl, thiadiazolyl, oxadiazolyl, pyridinyl, pyrimidyl, pyrazinyl, pyridazinyl, quinolyl, isoquniolyl, indazolyl, benzoxazolyl, benzofuryl, benzothiazolyl, indolizinyl, imidazopyridinyl, pyrazolyl, triazolyl, isothiazolyl, oxazolyl, tetrazolyl, benzimidazolyl, benzoxazolyl, benzothiazolyl, benzothiadiazolyl, benzoxadiazolyl, indolyl, tetrahydroindolyl, azaindolyl, imidazopyridyl, qunizaolinyl, purinyl, pyrrolo[2,3]pyrimidyl, pyrazolo[3,4]pyrimidyl or benzo(b)thienyl each of which is optionally substituted. Heteroaryl groups may be substituted or unsubstituted.

A heterocycloalkyl refers to a non-aromatic ring which contains one or more oxygen, nitrogen or sulfur (e.g., morpholine, piperidine, piperazine, pyrrolidine, and thiomorpholine). Heterocycloalkyl groups may be substituted or unsubstituted.

A primary amine group has the formula —NH2. A secondary amine group is a group having the formula —NHR, wherein R is an alkyl group, a modified alkyl group, or an aromatic group.

Hyphens, or dashes, are used at various points throughout this specification to indicate attachment, e.g. where two named groups are immediately adjacent a dash in the text, this indicates the two named groups are attached to each other. Similarly, a series of named groups with dashes between each of the named groups in the text indicates the named groups are attached to each other in the order shown. Also, a single named group adjacent a dash in the text indicates the named group is typically attached to some other, unnamed group. In some embodiments, the attachment indicated by a dash may be, e.g. a covalent bond between the adjacent named groups. In some other embodiments, the dash may indicate indirect attachment, i.e. with intervening groups between the named groups. At various points throughout the specification a group may be set forth in the text with or without an adjacent dash, (e.g. amido or amido-, further e.g. —OH or OH) where the context indicates the group is intended to be (or has the potential to be) bound to another group; in such cases, the identity of the group is denoted by the group name (whether or not there is an adjacent dash in the text). Note that where context indicates, a single group may be attached to more than one other group (e.g. the indicated group may have a substituent; further e.g. where a linkage is intended, such as linking groups).

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

Accordingly, in a first embodiment, the invention is directed to a method of producing a protein bioarray, wherein the method includes providing a substrate comprising a solid support and a surface modification layer bound to the solid support The surface modification layer includes a first moiety having the structure —Si—R¹ and a second moiety having the structure —Si-L-R², wherein R¹ is a chemically inert moiety selected from the group consisting of C₃ to C₃₀ alkyl and benzyl optionally substituted with 1 to 5 halogen atoms, L is a linking group, and R² is a chemically inert hydrophilic moiety. The method of producing the protein bioarray further includes providing at least two solutions, wherein each solution contains a probe protein. In the method, each of the at least two solutions is then deposited at its own discrete site on the substrate. The probe proteins deposited on the substrate become non-covalently bound to the substrate.

The present methods may be used to deposit solutions of probe proteins on surfaces of any of a variety of different substrates, including both flexible and rigid substrates. The substrate may take any of a variety of configurations ranging from simple to complex. Thus, the substrate could have a generally planar form, as for example a slide or plate configuration, such as a rectangular or square plate or disc. In many embodiments, the substrate will be shaped generally as a rectangular solid, having a length in the range about 4 mm to 1 m, usually about 4 mm to 600 mm, more usually about 4 mm to 400 mm; a width in the range about 4 mm to 1 m, usually about 4 mm to 500 mm and more usually about 4 mm to 400 mm; and a thickness in the range about 0.01 mm to 5.0 mm, usually from about 0.1 mm to 2 mm and more usually from about 0.2 to 1 mm. However, larger substrates can be used, particularly when such are cut after fabrication into smaller size substrates carrying a smaller total number of arrays. The substrates may be fabricated from any of a variety of materials. The solid support of the substrate may be any material that can provide physical support for the deposited material and endure the conditions of the deposition process and of any subsequent treatment or handling or processing that may be encountered in the use of the protein array. For flexible substrates, materials that the solid support may be made of include: nylon, both modified and unmodified, nitrocellulose, and the like, where a nylon membrane, as well as derivatives thereof, may be used. For rigid substrates, the solid support may be comprised of such materials as glass; fused silica; plastics (for example, polytetrafluoroethylene, polystyrene, polycarbonate, and blends thereof), titanium dioxide, and the like.

The modification layer may be bound to the solid support by any suitable method, particularly that disclosed by Lefkowitz et al. in U.S. Pat. No. 6,444,268, the teachings of such method incorporated herein by reference. Lefkowitz et al. teaches contacting the surface of a solid support with a derivatizing composition that contains a mixture of silanes, under reaction conditions effective to couple the silanes to the surface of the solid support via hydrophilic moieties present on the solid support surface. The hydrophilic moieties on the substrate surface are typically hydroxyl groups, carboxyl groups, thiol groups, and/or substituted or unsubstituted amino groups, although, typically, the reactive hydrophilic moieties are hydroxyl groups. The solid support may comprise any material that has a plurality of hydrophilic sites on its surface, or that can be treated or coated so as to have a plurality of such sites on its surface. Suitable materials include, but are not limited to, supports that are typically used for solid phase chemical synthesis, e.g., cross-linked polymeric materials (e.g., divinylbenzene styrene-based polymers), agarose (e.g., Sepharose™), dextran (e.g., Sephadex.R.™), cellulosic polymers, polyacrylamides, silica, glass (particularly controlled pore glass, or “CPG”), ceramics, and the like. The supports may be obtained commercially and used as is, or they may be treated or coated prior to functionalization.

The derivatizing composition contains two types of silanes, a first silane that may be represented as R¹—Si(R^{L}R^{x}R^{y}) and a second silane having the formula R²-L-Si(R^{L}R^{x}R^{y}). In these formulae, the R^{L}, which may be the same or different, are leaving groups, the R^{x} and R^{y}, which may be the same or different, are either lower alkyl or leaving groups like R^{L}, R¹ is a chemically inert moiety that upon binding to the substrate surface lowers the surface energy thereof, L is a linking group, and R² is a chemically inert hydrophilic moiety. Reaction of the substrate surface with the derivatizing composition is carried out under reaction conditions effective to couple the silanes to the surface hydrophilic moieties and thereby provide —Si—R¹ groups and —Si-L-R² groups on the substrate surface.

More specifically, the R^{L} moieties, which are leaving groups, are such that they enable binding of the silanes to the surface. Typically, the leaving groups are hydrolyzable so as to form a silanol linkage to surface hydroxyl groups. Examples of suitable leaving groups include, but are not limited to, halogen atoms, particularly chloro, and alkoxy moieties, particularly lower alkoxy moieties. The R^{x} and R^{y} are either lower alkyl, e.g., methyl, ethyl, isopropyl, n-propyl, t-butyl, or the like, or leaving groups as just described with respect to R^{L}. Thus, each type of silane will generally contain a trichlorosilyl functionality, a tri(lower)alkoxysilyl functionality such as trimethoxysilyl, mixed functionalities such as diisopropylchlorosilyl, dimethylchlorosilyl, ethyldichlorosilyl, methylethylchlorosilyl or the like.

The first silane is the derivatizing agent that reduces surface energy as desired, while the second silane provides the surface functionalization necessary for covalent attachment of an additional molecular moiety, e.g., a ligand, a monomer, an oligomer, etc. Thus, with respect to the first silane, coupling to the substrate yields surface —Si—R¹ groups as explained above, wherein R¹ is a chemically inert moiety that upon binding to the substrate surface lowers surface energy. By “chemically inert” is meant that R¹ will not be cleaved or form covalent bond with a protein when the functionalized substrate is used for its intended purpose, e.g. in forming the protein array as described herein, in performing binding assays, or the like. Typically, R¹ is an alkyl group, generally although not necessarily containing in the range of 3 to 30 carbon atoms, typically in the range of 4 to 20 carbon atoms, more typically in the range of 5 to 12 carbon atoms. R¹ may also be benzyl, either unsubstituted or substituted, with 1 to 5, typically 1 to 3, halogen, preferably fluoro, atoms.

The second silane, upon coupling, provides surface —Si-L-R² groups. Of course, if the R^{x} and R^{y} are not leaving groups, the surface moieties provided will actually be “—Si R^{x}R^{y}-L-R²” groups, which applicants intend to encompass by the more generic representation “—Si-L-R²”. R² is a chemically inert hydrophilic moiety. That is, R² may be a group such as hydroxyl, acetyl, carboxyl, amino, amide, methoxyl, ethoxyl, propoxyl, or the like. In particular embodiments, R² is —OCH₂CH₂)ₖ—H where k is an integer from 1 to about 10. L represents a linker that is generally an alkyl group having from about 3 to about 30 carbons. Normally, L is C₅ to C₃₀ alkyl, or more typically C₁₀ to C₁₈ alkyl.

The density of R² groups on the substrate surface, following reaction with the derivatizing composition, is determined by the relative proportions of the first and second silanes in the derivatizing composition. That is, a higher proportion of the second silane in the derivatizing composition will provide a greater density of R² groups, while a higher proportion of the first silane will give rise to a lower density of R² groups. Optimally, the first silane represents in the range of approximately 0.5 wt. % to 50 wt. % of the derivatization composition, preferably in the range of approximately 1.0 wt. % to 10 wt. % of the composition, while the second silane correspondingly represents in the range of approximately 50 wt. % to 99.5 wt. % of the derivatization composition, preferably in the range of approximately 90 wt. % to 99 wt. % of the composition.

Having provided a substrate as described above, the method of producing the protein bioarray further includes providing at least two solutions, wherein each solution contains a probe protein. In the method, each of the solutions is then deposited at its own discrete site on the substrate. The probe proteins deposited on the substrate become non-covalently bound to the substrate. In particular embodiments, at least twenty solutions are provided, each solution containing a probe protein. In some embodiments, at least 100 solutions are provided, each solution containing a probe protein. In still other embodiments, at least 250 solutions are provided, each solution containing a probe protein. In particular embodiments, up to about 1000 solutions may provided, each solution containing a probe protein. In some embodiments, up to about 5000 solutions may be provided, each solution containing a probe protein. In still other embodiments, up to about 25,000 solutions are provided, each solution containing a probe protein.

Each provided solution is deposited at a discrete site on the substrate, such that each solution is placed at its own discrete site. In certain embodiments, at least one solution contains a different probe protein than another of the solutions so that the features formed therefrom will display different probes. In certain embodiments, at least two solutions contain the same probe protein so that the features formed at the respective sites of the two solutions will display the same probe. In some embodiments, at least two solutions contain the same probe protein, but at different concentrations to result in the probe bound to the substrate at different concentrations at at least two discrete sites.

Any method effective to deliver the solutions to the substrate may be employed. Typical methods known in the art include pin-spotting methods, micropipetting methods, and inkjet methods. The quantity of solution delivered and the size of the site covered by the delivered solution will vary depending on design considerations. Inkjet methods will typically deliver droplets of the solutions to the substrate surface to produce a feature that is about 30 to about 150 micrometers in diameter. Spotting methods and micropipetting methods deliver more solution to the surface, forming larger features. Typical feature sizes are in the range from 100 micrometers to about 1000 micrometers in diameter, though embodiments outside these ranges may be accomplished and are within the scope of the invention. Protein concentration in the solution may be from about 25 micrograms total protein per milliliter, up to about 500 micrograms total protein per milliliter for inkjet deposition, and up to about 5 milligrams total protein per milliliter for other deposition methods, like contact printing.

In a typical embodiment, the solutions are allowed to dry on the substrate after deposition.

In some embodiments, the method further includes contacting the substrate surface with a blocking composition after the probe proteins are non-covalently bound to the substrate. In some embodiments the discrete sites on the substrate are separated by intervening space, and the blocking composition is generally washed over a surface of the substrate, thereby contacting the discrete spots and the intervening sites on the substrate. The blocking composition typically contains one or more blocking proteins, such that the blocking proteins typically non-covalently bind to portions of the substrate surface not already occupied by probe proteins. The binding of blocking proteins on the substrate surface tends to block sites/areas that non-specifically bind to proteins and would therefore contribute to noise in assays employing the protein array. The blocking composition typically includes one or more proteins selected from milk protein, casein, bovine serum albumin, fetal calf serum, or any other effective blocking agents. The concentration of protein in the blocking composition is typically in the range from about 1% to about 12%. The blocking occurs under conditions and for a time sufficient to result in binding of the blocking protein to the substrate, which results in low background signal (relative to binding of target protein by the probe proteins) during use of the protein array in a binding assay.

In an additional embodiment, the invention provides a protein array that includes a substrate comprising a solid support and a surface modification layer bound to the solid support. The surface modification layer includes a first moiety having the structure —Si—R¹ and a second moiety having the structure —Si-L-R², wherein R¹ is a chemically inert moiety selected from the group consisting of C₃ to C₃₀ alkyl and benzyl optionally substituted with 1 to 5 halogen atoms, L is a linking group, R₂ is a chemically inert hydrophilic moiety. In typical embodiments, R¹ is C₄ to C₂₀ alkyl, more typically C₅ to C₁₂ alkyl. In certain embodiments, the second moiety comprises from about 0.5% to about 99.5% of the modification layer, more typically from about 0.5% to about 30% of the modification layer. R² may be a group such as hydroxyl, acetyl, carboxyl, amino, amide, methoxyl, ethoxyl, propoxyl, or the like. In particular embodiments, R₂ is —(OCH₂CH₂)k-H where k is an integer from 1 to about 10. The protein array further includes a plurality of discrete sites on the substrate, each site having a probe protein bound thereto via non-covalent interaction. In particular embodiments, the protein array further includes blocking protein bound to the substrate.

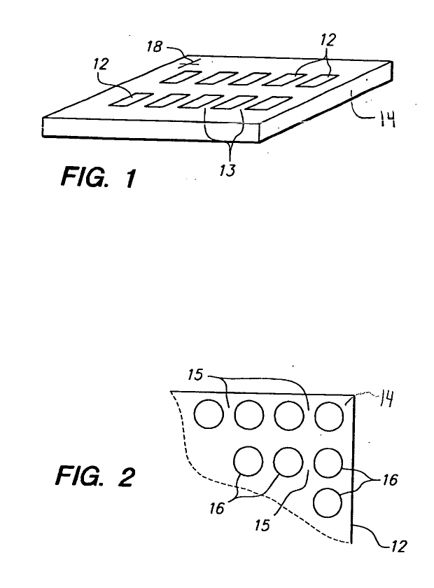

Referring now to FIGS. 1 and 2, the invention as described herein may be practiced to produce one or more arrays 12 (only some of which are shown in FIG. 1) across the surface of a single substrate 14, wherein the substrate comprises a solid support having a modification layer bound thereto. The arrays 12 produced on a given substrate need not be identical and some or all could be different. The surface of the substrate may include interarray areas 13 and may also include a fiducial 18. FIG. 2 depicts a single array 12 having multiple spots or features, 16 at discrete sites on the substrate 14. The features 16 may be separated by intervening space 15. An array 12 may contain any number of features, generally including at least tens of features, usually at least hundreds, more usually thousands, and as many as a hundred thousand or more features. All of the features 16 may be different, or some or all could be the same. Each region carries a predetermined protein or a predetermined mixture of proteins bound to the modification layer of the substrate by non-covalent bonding. The features of the array may be arranged in any desired pattern, e.g. organized rows and columns of features (for example, a grid of features across the substrate surface), a series of curvilinear rows across the substrate surface (for example, a series of concentric circles or semi-circles of features), and the like. In embodiments where very small feature sizes are desired, the density of features on the substrate may range from at least about ten features per square centimeter, or preferably at least about 35 features per square centimeter, or more preferably at least about 100 features per square centimeter and up to about 1000 features per square centimeter, or preferably up to about 10,000 features per square centimeter, or perhaps up to 100,000 features per square centimeter.

### EXAMPLES

The practice of the present invention will employ, unless otherwise indicated, conventional techniques of synthetic organic chemistry, biochemistry, molecular biology, and the like, which are within the skill of the art. Such techniques are explained fully in the literature.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. Standard temperature and pressure are defined as 20° C. and 1 atmosphere.

### Example 1

Preparation of Functionalized Surfaces

This example describes functionalization of a glass substrate with a derivatizing composition comprising 97.5% (wt) n-decyltricholorosilane (NTS) and 2.5% (wt) undecenyltrichlorosilane (UTS) followed by boration and oxidation to convert the terminal olefinic moiety of UTS to hydroxyl group.

(a) Silylation:

Under moisture-free conditions, 14 ml NTS and 0.4 ml UTS were added to 800 ml anhydrous toluene. The solution was mixed. Cleaned glass substrates were placed into 1 L reactor equipped for inert gas purging, heating and stirring. Purging was conducted for 30 min before NTS/UTS mixture was added to the reactor. The solution was heated to 100° C. for 4 hours under constant stirring while continuing to maintain moisture-free conditions. The silane solution was removed from the reactor and replaced with anhydrous toluene. This step was repeated twice.

The substrates were removed from reactor and rinsed rigorously with anhydrous toluene. The substrates were blown dry with clean inert gas and placed in a vacuum oven preheated to 150° C. and heated under vacuum for 1 hour. The substrates were then removed and allowed to cool to ambient temperature.

(b) Boration and Oxidation:

The silylated substrates were placed in a 1 liter reactor equipped with inert gas purging and stirring. The reactor was purged with moisture free inert gas for 30 mins before 800 ml of 1.0 M borane-tetrahydrofuran complex was transferred to the reactor. The substrates were incubated for 2 hours under constant stirring. The boration solution was removed and rinsed vigorously with appropriate solvents. The substrates were blown dry with clean inert gas.

To a 1 liter reactor equipped with stirring, 800 ml of 0.1 N NaOH in 30% aqueous hydrogen peroxide was added. The substrates were incubated for 10 mins under constant stirring. The substrates were removed and rinsed with appropriate solvents and blown dried by blown with clean inert gas.

### Example 2

Printing of Antibodies on Chemically Modified Substrates

The antibodies were diluted in a buffer compatible with thermal inkjet printing to a desired concentration (e.g. 250 ug/ml in the following example). The antibodies solutions were put in designated wells in a 96-well plate and then transferred by a robot to 384-flex plate. The antibody solution was then pushed up in flex plate and picked up by printing head under capillary action and vacuum. The antibody solutions were then printed as about 40 pL droplet onto each surface at designated area controlled by printing software.

The antibodies used herein were anti-human serum albumin (HSA) (Research Diagnostics, TRK4T24-15C7) anti-human transferrin (Research Diagnostics, TRK4T15-2A2), anti-human IgG1 (Research Diagnostics, TRK1G2-2C11), anti-human-light chain IgG1 (Research Diagnostics, TRK1K9-7A9) and many others. 1″×3″inch surface as prepared in the example 1 and Telechem super epoxy surface (Telechem, Sunnyvale, Calif.) were used.

### Example 3

Binding of Printed Antibodies with Cy3-Labeled Targets

(a) Preblock:

The printed slides were immersed in Casein Block (Pierce, catalog #37528) for 10 mins in a 25 mL polypropylene tube (Prolinx, VMT 2200-10) to remove unbound protein and minimize non-specific adsorption. A nutator (Boekel Model 2602SO) at setting of 12 rpm and was used for mixing of block solution. The slides were rinsed 3 times with PBS and once with DI water before drying with centrifuge at 1500 rpm (Beckman GPKR centrifuge) for 2 min.

(b) Incubate Printed Substrate with Cy3 Labeled Protein

HSA (Sigma A 8763), human-transferrin (Sigma T 3309) and human-IgG (Sigma I 2511) were labeled as described in Haab et al. (2001) Genome Biology, 2(2), 1-13. The labeled protein was diluted in buffer to a concentration of 1 ug/ml for Cy3-HSA and Cy3-human-transferrin, 2 ug/ml for Cy3-human IgG. 200 uL of diluted sample was applied to Agilent Sure Hyb gasket slide. The printed slides were then assembled using Agilent SureHyb chamber.

The printed arrays were incubated for 2 hr at room temperature using gentle rotation at 4 rpm in a Hybridization Incubator (Robbins Scientific, Model 400). After incubation, the slides were washed twice in PBS containing 0.05% Tween-20 (PBST) for 10 min and once in PBS for 10 min. The slides were then rinsed with deionized (DI) water and spun to dryness in a centrifuge at 1500 rpm for 2 min.

(c) Fluorescence Scan

The processed slides were scanned using the Agilent DNA Microarray Scanner. The scan resolution was chosen to be 5 um.

(d) Data Analysis and Results:

Agilent Feature Extraction software (G2567AA, Version 7.1.1) was used for data analysis. The results presented as follows are the average of 24 spots per array for each antibody at specific condition.

The experimental results showed that the substrate prepared as described in these Examples (Example 1) gave 3× to 5× higher signals than Telechem Epoxy slides under identical experimental conditions. This is due to more efficient antibody deposition, better orientation to bind target and/or antibody activity preservation.

### Example 4

Background Signal on Different Chemically Modified Surfaces

(a) Preblock:

PLL was prepared as described in reference with minor modifications (Haab et al. (2001) Genome Biology, 2(2), 1-13). Substrates were prepared as described in Example 1. Super Epoxy was purchased from Telechem (Sunnyvale, Calif.). The slides were immersed in Casein Block (Pierce, catalog #37528) for 10 mins in a 25 mL polypropylene tube (Prolinx, VMT 2200-10) to remove unbound protein and minimize non-specific adsorption. A nutator (Boekel Model 2602SO) at setting of 12 rpm and was used for mixing. The slides were rinsed 3 times with PBS and once with water before drying with centrifuge at 1500 rpm (Beckman GPKR centrifuge) for 2 min.

(b) Incubation with Cy3 Labeled Human Serum Proteins

1 mg of human serum protein (Sigma S2257) was labeled with reactive Cy3 (Amersham Q13108) under protocol described in the reference with minor modification (Haab et al. (2001) Genome Biology, 2(2), 1-13). The labeled protein (3500 ug/ml) was diluted to 350 ug/ml in Casein Block (Pierce, catalog #37528). 200 uL sample was applied to Agilent SureHyb gasket slide. The preblocked three different surfaces were assembled using Agilent SureHyb chamber.

(c) Fluorescence Scan and Results

The processed slides were scanned using an Agilent DNA Microarray Scanner. The scan resolution was chosen to be 10 um.

The results in the following table showed that substrates prepared as per Example 1, above, gave the least background signal caused by the non-specific adsorption of target mixture. This demonstrates another advantage of using hydrophobic surface for protein array. The preblock is more efficient on hydrophobic surface. This makes the hydrophobic surface more effective in repelling labeled proteins binding onto surface non-specifically.

| Surface | Background signal (cts) |
|---|---|
| Substrates per Example 1 | 500-600 |
| Telechem Epoxy | ˜4000 |
| PLL | 20,000-25,000 |

Lefkowitz et al. (U.S. Pat. No. 6,258,454 B1; U.S. Pat. No. 6,444,268 B2) have described a general method of preparing surfaces having a self-assembled hydrophobic layer using a mixture of two silanes. Lefkowitz further teaches that, by adjusting the ratio of the two silanes, the surface functionality of the resulting surfaces may be adjusted. We have now found that such surfaces are surprisingly effective as surfaces for protein deposition. The hydrophobic nature of the self-assembled layer binds protein by strong hydrophobic-hydrophobic interactions (and/or other non-covalent interactions). The functionality on the surface can be used to adjust surface energy and to provide hydrogen-bonding sites, which in turn would increase the van der Waal interaction of proteins with modified surface. Proteins can form a tightly bound layer on hydrophobic surfaces and some of them retain their target binding capability. Dong et al. (2000) Analytical Chemistry, 72: 2371-76; Davies, et al. Langmuir, 10 (8), 2654-61. Non-specific binding properties of the proteins on hydrophobic surface provide another advantage. The unspotted area can be sufficiently blocked by blocking proteins. This will decrease the background caused by labeled target proteins that non-specifically bind to chemically modified surfaces.

The present invention relates to the deposition of proteins on a hydrophobic surface having a modification layer comprising a mixture of two silane group. The deposited proteins are bound to the surface by non-covalent interactions, e.g. through van der Waals interactions. The composition of different silanes will adjust surface energy, probe density as well as interaction efficiency.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties, provided that, if there is any conflict in definitions, the definitions herein shall control.

1. A method of producing a protein bioarray comprising:
a) providing a substrate comprising a solid support and a surface modification layer bound to the solid support, the surface modification layer comprising at least a first moiety having the structure —Si—R¹ and a second moiety having the structure —Si-L-R^{2,} wherein R¹ is a chemically inert moiety selected from the group consisting of C₃ to C₃₀ alkyl and benzyl optionally substituted with 1 to 5 halogen atoms, L is a linking group, R² is a chemically inert hydrophilic moiety,
b) providing at least two solutions, each solution comprising a probe protein, and
c) depositing the solutions provided in step b) onto discrete sites on the substrate, each solution being deposited onto its own discrete site, wherein each probe protein becomes non-covalently attached to the substrate at its respective discrete site.

2. The method of claim 1, further comprising drying the substrate after depositing the solutions.

3. The method of claim 1, further comprising, after step c),
d) contacting the substrate with a blocking composition comprising a blocking protein, wherein the blocking protein becomes non-covalently attached to the substrate

4. The method of claim 3, wherein the discrete sites are separated by intervening areas, and the blocking protein becomes non-covalently attached to the substrate at the intervening areas and at the discrete sites.

5. The method of claim 3, wherein the blocking composition comprises a plurality of blocking proteins.

6. The method of claim 5, wherein the plurality of blocking proteins are selected to provide low background signal relative to binding of target protein by the probe proteins.

7. The method of claim 1, wherein at least one solution provided in step b) comprises a probe protein that is different from at least one other probe protein in another solution provided in step b).

8. The method of claim 1, wherein least fifty solutions are provided in step b).

9. The method of claim 1, wherein least 250 solutions are provided in step b).

10. The method of claim 1,wherein depositing the solutions comprises using an inkjet apparatus to deliver one or more droplets of each solution to its respective discrete site.

11. A protein bioarray comprising
a substrate comprising a solid support and a surface modification layer bound to the solid support, the surface modification layer comprising at least a first moiety having the structure —Si—R¹ and a second moiety having the structure —Si-L-R², wherein R¹ is a chemically inert moiety selected from the group consisting of C₃ to C₃₀ alkyl and benzyl optionally substituted with 1 to 5 halogen atoms, L is a linking group, R² is a chemically inert hydrophilic moiety;
a plurality of discrete sites on the substrate, each site having a probe protein bound thereto via non-covalent interaction.

12. The protein bioarray of claim 11, further comprising intervening areas between the discrete sites.

13. The protein bioarray of claim 11, further comprising a blocking protein bound to the substrate.

14. The protein bioarray of claim 11, wherein each discrete site is in the range from 30 to 150 micrometers in diameter.

15. The protein bioarray of claim 11, wherein the solid support comprises a material selected from glass; fused silica; plastic, polytetrafluoroethylene, polystyrene, polycarbonate, ceramic, titanium dioxide.

16. The protein bioarray of claim 11, wherein the second moiety comprises from about 0.5% to about 99.5% of the modification layer.

17. The protein bioarray of claim 11, wherein the second moiety comprises from about 0.5% to about 30% of the modification layer.

18. The protein bioarray of claim 11, wherein R² is selected from hydroxyl, acetyl, carboxyl, amino, amide, methoxyl, ethoxyl, propoxyl, and —OCH₂CH₂)ₖ—H where k is an integer from 1 to about 10.
